(19) United States
(12) Patent Application Publication
(10) Pub. No.: US 2026/0283832 A1
(43) Pub. Date: Sep. 24, 2026

(54) ABDOMINAL SUPPORT BELT AND BACK SUPPORT MEMBER

(71) Applicant: Shenzhen Lute Jiacheng Supply Chain Management Co., Ltd., Shenzhen (CN)

(72) Inventor: Zijuan Qi, Shenzhen (CN)

(21) Appl. No.: 19/097,834

(22) Filed: Apr. 1, 2025

(30) Foreign Application Priority Data
Mar. 20, 2025 (CN) 202520503637.6

(51) Int. Cl. A61F 5/02; A61F 5/03

(52) U.S. Cl. CPC A61F 5/028; A61F 5/03

(57) ABSTRACT

The present application discloses an abdominal support belt and a back support member. The abdominal support belt comprises: a waist support portion configured to extend across a waist area of a user and along both lateral sides of the user when worn; a back support member disposed on the waist support portion, the back support member including at least one support strip extending along a height direction of the abdominal support belt when worn, the support strip being progressively curved toward the user's back from both ends to a central portion thereof when the abdominal support belt is worn; and an abdominal support portion connected to the waist support portion, configured to be positioned over an abdominal area of the user when worn.

### CROSS-REFERENCE TO RELATED APPLICATIONS

The present application claims priority to Chinese Patent Application No. 202520503637.6, filed on Mar. 20, 2025, the entire contents of which are incorporated herein by reference.

### TECHNICAL FIELD

The present application relates to the technical field of maternal and baby products, particularly to an abdominal support belt and back support member.

### BACKGROUND

The abdominal support belt, as an assistive device widely used by pregnant women and individuals requiring lumbar support, primarily functions to alleviate back pressure caused by changes in body center of gravity or muscle fatigue through wrapping and supporting the waist and abdominal regions, thereby improving postural stability and enhancing wearing comfort. Traditional designs of abdominal support belts typically focus on the combination of a waist support portion and an abdominal support portion, utilizing elastic materials or adjustable straps to achieve physical fixation and localized support for the waist and abdomen. For example, high-elasticity fabrics may form a looped wrap, or adjustable devices such as hook-and-loop fasteners or buckles may be used to accommodate different body types.

While such designs can provide basic support in static scenarios, they still face significant challenges in practical use. Traditional back support members in abdominal support belts are predominantly planar in design, making it difficult to cover the deep muscle groups on both sides of the spine, resulting in a support effect characterized by localized strength but overall weakness. Traditional back support members often rely on the elastic deformation of materials to provide passive pressure, lacking adaptability to the natural curvature of the human spine and dynamic movements. Particularly when users bend, turn, or stand for extended periods, the transmission path of support force is prone to deviation, causing pressure to concentrate on localized areas of the lumbar spine. This not only reduces support efficiency but may also exacerbate muscle fatigue. In traditional designs, reinforcement methods for the back are often indirect, such as thickening the waist pad or adding horizontal straps. While such solutions may temporarily relieve back pressure, they lack conformity design to the physiological curves of the spine, leading to misalignment between the support member and the body's contact surface. In some cases, excessive compression from rigid materials may even cause discomfort during wear. Additionally, existing abdominal support belts have limitations in breathability and lightweight design. The stacking of multiple material layers or the inclusion of rigid support members may increase heat retention, further compromising the feasibility of prolonged wear.

### SUMMARY

In view of the shortcomings of the prior art, embodiments of the present application provide an abdominal support belt and a back support member, which can enhance the back support effect of the abdominal support belt.

To address the above technical problems, one embodiment of the present application provides an abdominal support belt. The abdominal support belt comprises: a waist support portion configured to extend across a waist area of a user and along both lateral sides of the user when worn; a back support member disposed on the waist support portion, the back support member including at least one support strip extending along a height direction of the abdominal support belt when worn, the support strip being progressively curved toward the user's back from both ends to a central portion thereof when the abdominal support belt is worn; and an abdominal support portion connected to the waist support portion, configured to be positioned over an abdominal area of the user when worn.

In one embodiment, the back support member comprises at least a first support strip and a second support strip, the first support strip and the second support strip being positioned on opposite lateral sides of the user's spine when worn, the first support strip being progressively curved from both ends toward a central portion in a direction approaching the spine when the abdominal support belt is worn, and the second support strip being progressively curved from both ends toward a central portion in a direction approaching the spine when the abdominal support belt is worn.

In one embodiment, the support strip has a thickness that gradually decreases from a central portion to both ends thereof.

In one embodiment, the back support member further comprises a connecting strip bridging adjacent support strips.

In one embodiment, the connecting strip includes at least a first connecting strip and a second connecting strip, the first connecting strip and the second connecting strip being arranged at different positions along a height direction of the abdominal support belt when worn.

In one embodiment, the back support member is embedded within the waist support portion.

In one embodiment, the waist support portion is at least partially made of a sheet material, the sheet material comprising an outer layer, an inner layer, and an adhesive layer interposed between the outer and inner layers.

In one embodiment, the adhesive layer comprises a first adhesive sublayer, a cushioning layer, and a second adhesive sublayer sequentially stacked from the inner layer toward the outer layer.

In one embodiment, the back support member is embedded within the cushioning layer.

In one embodiment, the cushioning layer comprises a sponge layer.

In one embodiment, the inner layer is provided with ventilation holes.

In one embodiment, the abdominal support portion is configured to overlay a lower abdominal area of the user when worn.

In one embodiment, the back support member is disposed on an exterior surface of the waist support portion.

In one embodiment, the support strip has an arcuate upper edge.

In one embodiment, the support strip has an arcuate lower edge.

To address the above technical problems, one embodiment of the present application provides a back support member for an abdominal support belt. The back support member comprises at least one support strip extending along a height direction of the abdominal support belt when worn, the support strip being progressively curved toward the user's back from both ends to a central portion thereof when the abdominal support belt is worn.

In one embodiment, the back support member comprises at least a first support strip and a second support strip, the first support strip and the second support strip being positioned on opposite lateral sides of the user's spine when worn, the first support strip being progressively curved from both ends toward a central portion in a direction approaching the spine when the abdominal support belt is worn, and the second support strip being progressively curved from both ends toward a central portion in a direction approaching the spine when the abdominal support belt is worn.

In one embodiment, the support strip has a thickness that gradually decreases from a central portion to both ends thereof.

In one embodiment, the back support member further comprises a connecting strip bridging adjacent support strips.

In one embodiment, the connecting strip includes at least a first connecting strip and a second connecting strip, the first connecting strip and the second connecting strip being arranged at different positions along a height direction of the abdominal support belt when worn.

To address the above technical problems, one embodiment of the present application provides an abdominal support belt. The abdominal support belt comprises: a waist support portion configured to extend across a waist area of a user and along both lateral sides of the user when worn; and an abdominal support portion connected to the waist support portion, configured to be positioned over an abdominal area of the user when worn. The abdominal support portion includes a first strap and a second strap respectively extending along both lateral sides of the waist support portion, the first strap and the second strap being detachably connected, and the lengths of the first strap and the second strap being unequal.

In one embodiment, the abdominal support belt is at least partially made of a sheet material, the sheet material comprising an outer layer, an inner layer, and an adhesive layer interposed between the outer and inner layers.

In one embodiment, the adhesive layer comprises a first adhesive sublayer, a cushioning layer, and a second adhesive sublayer sequentially stacked from the inner layer toward the outer layer.

In one embodiment, the first adhesive sublayer and the second adhesive sublayer are adhesive layers.

In one embodiment, the cushioning layer comprises a sponge layer.

In one embodiment, the inner layer is provided with ventilation holes.

In one embodiment, the length of the first strap is greater than the length of the second strap.

In one embodiment, the first strap comprises a side panel and an abdominal panel, the side panel extending along a lateral side of the user's abdomen when worn, the abdominal panel extending along the user's abdomen when worn, and the thickness of the abdominal panel being greater than the thickness of the side panel.

In one embodiment, the side panel and the abdominal panel are formed by stitching.

In one embodiment, the second strap is provided with a lifting handle.

In one embodiment, the waist support portion is embedded with a back support member, the hardness of the back support member being greater than the hardness of other portions of the waist support portion.

In one embodiment, the back support member comprises a plurality of support strips arranged at intervals.

In one embodiment, the support strips extend along a height direction of the user when the abdominal support belt is worn, and the thickness of the support strips gradually decreases from a central portion to both ends thereof.

In one embodiment, the support strips have arcuate upper edges.

In one embodiment, a lower edge of the waist support portion is linear at least at a position corresponding to the user's back.

In one embodiment, the first strap extends across the user's abdominal area when worn and is detachably connected to the second strap at a lateral side of the user.

In one embodiment, a width of a central portion of the abdominal panel is greater than widths of both ends thereof.

In one embodiment, a lower edge of the first strap extends obliquely upward from the abdominal area to a junction where the first strap and the second strap are connected when worn.

In one embodiment, a lower edge of the second strap extends obliquely downward from the waist area to a junction where the first strap and the second strap are connected when worn.

In one embodiment, the first strap and the second strap are connected by hook-and-loop fasteners, one of the first strap and the second strap being provided with a loop surface of the hook-and-loop fastener, and the other being provided with a hook surface of the hook-and-loop fastener.

In one embodiment, the hook-and-loop fastener on the second strap has a certain length, allowing the detachable connection position between the first strap and the second strap to be adjustable.

In one embodiment, the hook-and-loop fastener is stitched onto the first strap or the second strap by sewing threads, the sewing threads comprising peripheral sewing threads located at edge regions of the hook-and-loop fastener and intermediate sewing threads located at a central region of the hook-and-loop fastener.

To address the above technical problems, another embodiment of the present application provides an abdominal support belt. The abdominal support belt comprises: a waist support portion configured to extend across a waist area of a user and along both lateral sides of the user when worn; and an abdominal support portion connected to the waist support portion, configured to be positioned over an abdominal area of the user when worn. The abdominal support portion includes a first strap and a second strap respectively extending along both lateral sides of the waist support portion, the first strap and the second strap being detachably connected, and the first strap extending across the user's abdominal area when worn and being detachably connected to the second strap at a lateral side of the user.

In one embodiment, the abdominal support belt is at least partially made of a sheet material, the sheet material comprising an outer layer, an inner layer, and an adhesive layer interposed between the outer and inner layers.

In one embodiment, the adhesive layer comprises a first adhesive sublayer, a cushioning layer, and a second adhesive sublayer sequentially stacked from the inner layer toward the outer layer.

In one embodiment, the first adhesive sublayer and the second adhesive sublayer are adhesive layers.

In one embodiment, the cushioning layer comprises a sponge layer.

In one embodiment, the inner layer is provided with ventilation holes.

In one embodiment, the length of the first strap is greater than the length of the second strap.

In one embodiment, the first strap comprises a side panel and an abdominal panel, the side panel extending along a lateral side of the user's abdomen when worn, the abdominal panel extending along the user's abdomen when worn, and the thickness of the abdominal panel being greater than the thickness of the side panel.

In one embodiment, the side panel and the abdominal panel are formed by stitching.

In one embodiment, the second strap is provided with a lifting handle.

In one embodiment, the waist support portion is embedded with a back support member, the hardness of the back support member being greater than the hardness of other portions of the waist support portion.

In one embodiment, the back support member comprises a plurality of support strips arranged at intervals.

In one embodiment, the support strips extend along a height direction of the user when the abdominal support belt is worn, and the thickness of the support strips gradually decreases from a central portion to both ends thereof.

In one embodiment, the support strips have arcuate upper edges.

In one embodiment, a lower edge of the waist support portion is linear at least at a position corresponding to the user's back.

In one embodiment, a width of a central portion of the abdominal panel is greater than widths of both ends thereof.

In one embodiment, a lower edge of the first strap extends obliquely upward from the abdominal area to a junction where the first strap and the second strap are connected when worn.

In one embodiment, a lower edge of the second strap extends obliquely downward from the waist area to a junction where the first strap and the second strap are connected when worn.

In one embodiment, the first strap and the second strap are connected by hook-and-loop fasteners, one of the first strap and the second strap being provided with a loop surface of the hook-and-loop fastener, and the other being provided with a hook surface of the hook-and-loop fastener.

In one embodiment, the hook-and-loop fastener on the second strap has a certain length, allowing the detachable connection position between the first strap and the second strap to be adjustable.

In one embodiment, the hook-and-loop fastener is stitched onto the first strap or the second strap by sewing threads, the sewing threads comprising peripheral sewing threads located at edge regions of the hook-and-loop fastener and intermediate sewing threads located at a central region of the hook-and-loop fastener.

The abdominal support belt according to embodiments of the present application comprises: a waist support portion configured to extend across a waist area of a user and along both lateral sides of the user when worn; a back support member disposed on the waist support portion, the back support member including at least one support strip extending along a height direction of the abdominal support belt when worn, the support strip being progressively curved toward the user's back from both ends to a central portion thereof when the abdominal support belt is worn; and an abdominal support portion connected to the waist support portion, configured to be positioned over an abdominal area of the user when worn. By optimizing the design of the back support member, the abdominal support belt better conforms to the user's back curvature, distributes pressure, and provides dynamic adaptive support, thereby improving wearing comfort and support effectiveness.

### BRIEF DESCRIPTION OF THE DRAWINGS

To more clearly illustrate the technical solutions in the embodiments of the present application or the prior art, the following provides a brief introduction to the accompanying drawings used in the description of the embodiments or the prior art. Obviously, the drawings in the following description are only some embodiments of the present application. For those of ordinary skill in the art, other drawings can be obtained based on the structures shown in these drawings without creative effort.

FIG. 1 is a schematic structural diagram of the abdominal support belt in a worn state according to an embodiment of the present application, viewed from a first perspective;

FIG. 2 is a schematic structural diagram of the back support member according to an embodiment of the present application, viewed from a first perspective;

FIG. 3 is a schematic structural diagram of the back support member according to an embodiment of the present application, viewed from a second perspective;

FIG. 4 is a schematic structural diagram of the abdominal support belt in an unfolded state according to an embodiment of the present application;

FIG. 5 is a schematic structural diagram of the abdominal support belt in a worn state according to an embodiment of the present application;

FIG. 6 is a schematic diagram of the layered structure of a sheet material according to an embodiment of the present application;

FIG. 7 is a schematic diagram of the layered structure of a sheet material according to another embodiment of the present application.

The implementation, functional features, and advantages of the present application will be further described in conjunction with the embodiments and with reference to the accompanying drawings.

### DETAILED DESCRIPTION OF THE EMBODIMENTS

In the present application, the terms “arranged,” “provided with,” and “connected” should be understood broadly. For example, a connection may be fixed, detachable, or integrally constructed; it may be mechanical or electrical; it may be direct or indirectly connected through an intermediary, or it may refer to internal communication between two devices, components, or constituent parts. Those of ordinary skill in the art can understand the specific meanings of these terms in the context of the present application based on specific circumstances.

The terms “center,” “longitudinal,” “transverse,” “length,” “width,” “thickness,” “upper,” “lower,” “front,” “rear,” “left,” “right,” “vertical,” “horizontal,” “top,” “bottom,” “inner,” “outer,” “radial,” and “circumferential” are used to describe orientations or positional relationships based on the orientations or positional relationships shown in the accompanying drawings. These terms are used solely for the purpose of facilitating the description of the present application and simplifying the description, and do not indicate or imply that the referenced devices or components must have a specific orientation or be constructed and operated in a specific orientation. Therefore, these terms should not be construed as limiting the present application.

Furthermore, the terms “first” and “second” are used for descriptive purposes only and should not be construed as indicating or implying relative importance or implicitly specifying the quantity of the indicated technical features. Thus, features defined by “first” or “second” may explicitly or implicitly include at least one such feature. In the description of the present application, the term “plurality” means at least two, such as two, three, etc., unless explicitly stated otherwise.

Additionally, some of the above terms may be used to indicate meanings other than orientation or positional relationships. For example, the term “upper” may, in some cases, also be used to indicate a certain dependency or connection relationship. Those of ordinary skill in the art can understand the specific meanings of these terms in the context of the present application based on specific circumstances.

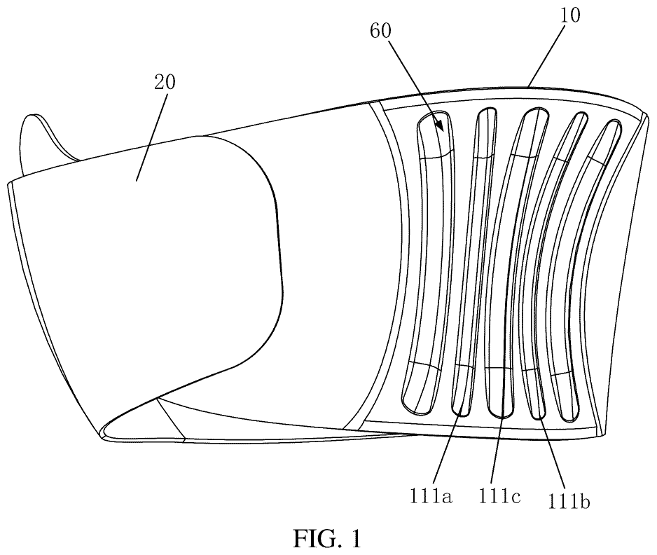

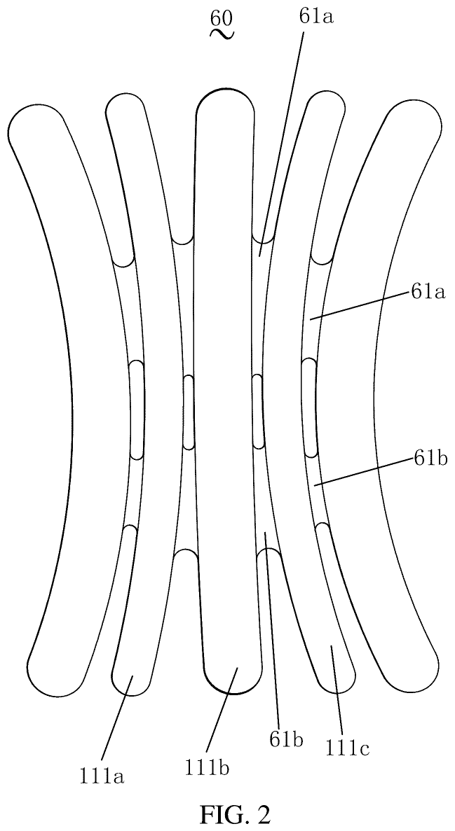

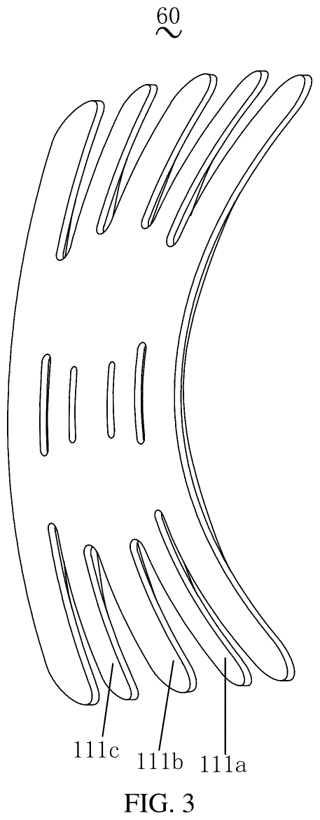

Please refer to FIG. 1 to FIG. 3. FIG. 1 is a schematic structural diagram of the abdominal support belt in a worn state according to an embodiment of the present application, viewed from a first perspective. FIG. 2 is a schematic structural diagram of the back support member according to an embodiment of the present application, viewed from a first perspective. FIG. 3 is a schematic structural diagram of the back support member according to an embodiment of the present application, viewed from a second perspective.

As shown in FIG. 1, the abdominal support belt according to an embodiment of the present application comprises: a waist support portion 10, an abdominal support portion 20, and a back support member 60.

The waist support portion 10 is configured to extend across a waist area of a user and along both lateral sides of the user when worn.

As shown in FIG. 1 to FIG. 3, the back support member 60 is disposed on the waist support portion 10. The back support member 60 may be embedded within the waist support portion 10 or disposed on an exterior surface of the waist support portion 10, which is not limited in the embodiments of the present application. The hardness of the back support member 60 is greater than that of the waist support portion 10 and the abdominal support portion 20. The back support member 60 is made of a rigid material and has elasticity. Optionally, the back support member 60 is made of TPU (Thermoplastic Polyurethane), rigid silicone, or carbon fiber composite material.

As shown in FIG. 1 to FIG. 3, the back support member 60 comprises at least one support strip 111a, 111b, 111c. The support strips 111a, 111b, 111c extend along a height direction of the user when the abdominal support belt is worn, and the support strips 111a, 111b, 111c are progressively curved toward the user's back from both ends to a central portion thereof when the abdominal support belt is worn. The support strips 111a, 111b, 111c are progressively curved from both ends toward the central portion in a direction approaching the user's back, allowing them to closely conform to the natural curvature of the user's spine, particularly the muscle groups on both sides of the spine. This design avoids the uneven distribution of support force caused by traditional planar back support members, ensuring that the support force is precisely applied to key areas of the back and effectively alleviating spinal pressure. Although the drawings show multiple support strips, it should be understood that the number of support strips may be one or two, which is not limited in the embodiments of the present application.

The abdominal support portion 20 is connected to the waist support portion 10 and is configured to be positioned over an abdominal area of the user when worn.

As shown in FIG. 1 to FIG. 3, in one embodiment, the back support member 60 comprises at least a first support strip 111a and a second support strip 111b. The first support strip 111a and the second support strip 111b are positioned on opposite lateral sides of the user's spine when the abdominal support belt is worn. The first support strip 111a is progressively curved from both ends toward a central portion in a direction approaching the spine when the abdominal support belt is worn, and the second support strip 111b is progressively curved from both ends toward a central portion in a direction approaching the spine when the abdominal support belt is worn. The support strip 111c extends substantially along the direction of the user's spine when the abdominal support belt is worn. The first support strip 111a and the second support strip 111b are respectively located on both sides of the spine, allowing them to closely conform to the natural curvature of the spine and provide symmetrical and evenly distributed support force. This design effectively disperses the pressure on the spine, reducing the burden on the spine and its surrounding muscle groups, making it particularly suitable for individuals who need to stand or move for extended periods.

As shown in FIG. 1 to FIG. 3, in one embodiment, the support strips 111a, 111b, 111c have a thickness that gradually decreases from a central portion to both ends. The thicker central portion of the support strips provides the main support force, ensuring effective support for the spine and its surrounding muscle groups, while the gradually thinning design at both ends achieves a smooth transition of the support force. This design avoids localized pressure concentration caused by abrupt changes in thickness, evenly distributes the support force, reduces pressure on the skin, and significantly improves wearing comfort. The thinning design at both ends of the support strips makes the edges of the abdominal support belt softer and lighter, reducing pressure and friction when in contact with the skin. This design is particularly suitable for long-term wear, effectively avoiding skin irritation caused by the edges of traditional support members and significantly enhancing skin-friendly comfort. The thinning design at both ends of the support strips also reduces the overall weight of the abdominal support belt, making it more lightweight and suitable for prolonged wear. This design is especially suitable for pregnant women, postpartum women, and individuals requiring lumbar support, significantly improving the wearing experience.

In one embodiment, the back support member 60 further comprises connecting strips 61a, 61b, which bridge adjacent support strips 111a, 111b. In one embodiment, a connecting strip is provided between every two adjacent support strips, thereby integrating the entire back support member 60 into a single unit. The connecting strips serve to enhance the overall support strength and stability, while also facilitating the integral injection molding of the back support member 60.

In one embodiment, the connecting strips 61a, 61b comprise at least a first connecting strip 61a and a second connecting strip 61b, which are arranged at different positions along the height direction of the abdominal support belt when worn.

In one embodiment, the back support member 60 is embedded within the waist support portion 10. By embedding the back support member 60 within the waist support portion 10, direct contact between the back support member 60 and the user's body can be avoided, and the waist support portion 10 provides a certain cushioning effect.

As shown in FIG. 6, in one embodiment, the waist support portion 10 is at least partially made of a sheet material 30. The sheet material 30 comprises an outer layer 31, an inner layer 32, and an adhesive layer 33 interposed between the outer layer 31 and the inner layer 32.

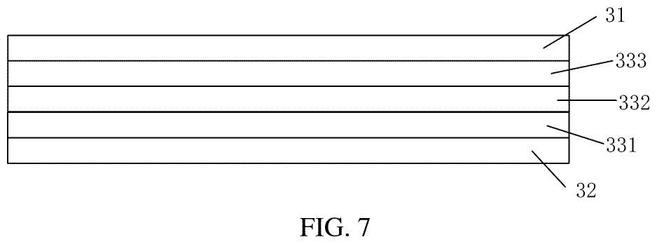

As shown in FIG. 7, in one embodiment, the adhesive layer 33 comprises a first adhesive sublayer 331, a cushioning layer 332, and a second adhesive sublayer 333 sequentially stacked from the inner layer 32 toward the outer layer 31.

In one embodiment, the back support member (not shown in the figure) is embedded within the cushioning layer 333. By embedding the back support member in the cushioning layer, the cushioning effect of the waist support portion 10 on the back support member is enhanced.

In one embodiment, the cushioning layer 333 is a sponge layer. The sponge layer has excellent elasticity and softness, providing comfortable cushioning during wear and reducing pressure on the waist and back. At the same time, the lightweight nature of the sponge layer does not increase the overall weight of the abdominal support belt, making it suitable for prolonged wear. Additionally, the sponge layer has excellent breathability, effectively wicking away sweat and keeping the skin dry, further enhancing wearing comfort.

In one embodiment, the inner layer 32 is provided with ventilation holes. The design of ventilation holes significantly improves the breathability of the abdominal support belt, promoting air circulation and avoiding discomfort caused by prolonged wear, such as heat buildup. The distribution of ventilation holes can be optimized according to the user's needs, ensuring optimal ventilation in key areas (e.g., the waist and back). This design is particularly suitable for use in hot environments or during high-intensity activities, significantly improving user experience.

In one embodiment, the abdominal support portion 20 is configured to be positioned over a lower abdominal area of the user when worn. This design provides precise support to the user's lower abdomen, making it particularly suitable for pregnant women or individuals requiring additional abdominal support. By concentrating the support force on the lower abdominal area, it effectively alleviates pressure on the waist and back caused by increased abdominal weight and improves postural stability. Additionally, this design provides extra abdominal support for postpartum women, aiding in the recovery of abdominal muscle strength.

In one embodiment, the upper edges of the support strips 111a, 111b, 111c are arcuate. The arcuate design reduces contact pressure between the support strips and the user's back, avoiding friction or pressure from the edges on the skin. This design is particularly suitable for long-term wear, significantly enhancing skin-friendly comfort. Furthermore, the arcuate edges enhance the aesthetic appeal of the support strips, giving the abdominal support belt a more streamlined and natural appearance.

In one embodiment, the lower edges of the support strips 111a, 111b, 111c are arcuate. The arcuate design of the lower edges complements the upper edges, further optimizing the fit between the support strips and the user's body. This design prevents the lower edges of the support strips from exerting pressure on the waist, reducing the sense of discomfort during wear. At the same time, the arcuate edges enhance the dynamic adaptability of the support strips, making them more flexible when the user bends or turns, thereby improving the overall wearing experience.

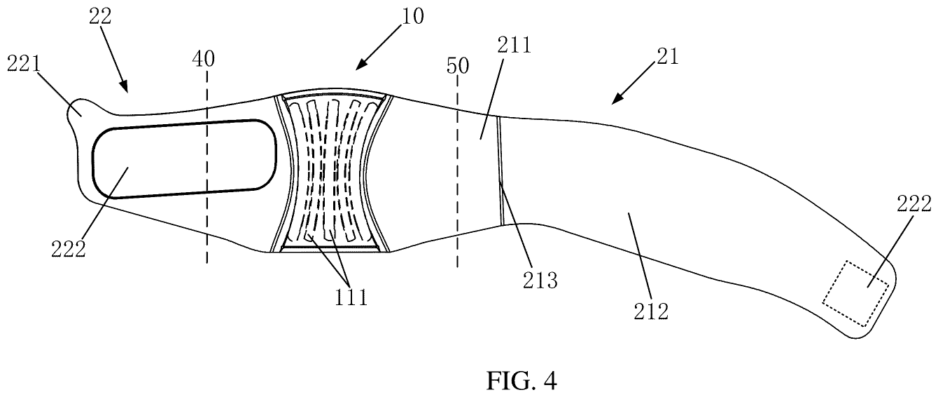

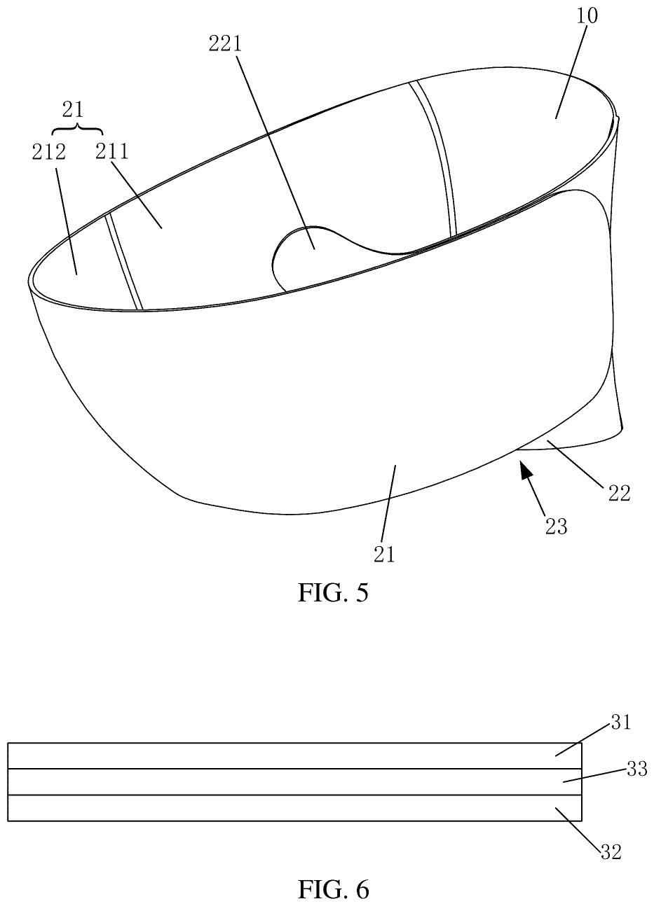

Please refer to FIG. 4 and FIG. 5. FIG. 4 is a schematic structural diagram of the abdominal support belt in an unfolded state according to an embodiment of the present application. FIG. 5 is a schematic structural diagram of the abdominal support belt in a worn state, viewed from a second perspective, according to an embodiment of the present application.

The abdominal support belt according to an embodiment of the present application comprises: a waist support portion 10 and an abdominal support portion (not labeled).

The waist support portion 10 is configured to extend across a waist area of a user and along both lateral sides of the user when worn.

As shown in FIG. 4 and FIG. 5, the abdominal support portion is connected to the waist support portion 10 and is configured to be positioned over an abdominal area of the user when worn. The abdominal support portion includes a first strap 21 and a second strap 22 respectively extending along both lateral sides of the waist support portion 10. The first strap 21 and the second strap 22 are detachably connected, and the lengths of the first strap 21 and the second strap 22 are unequal.

As shown in FIG. 5, the abdominal support portion and the waist support portion 10 may be integrally connected, cut from the same sheet material, or formed by stitching or detachable connections. Dotted line 40 and dotted line 50 approximately correspond to the positions of the left and right midlines of the human body when worn, respectively. For example, dotted line 40 corresponds to the left midline of the human body, and dotted line 50 corresponds to the right midline of the human body. The area to the left of dotted line 40 is the second strap 22, the area between dotted line 40 and dotted line 50 is the waist support portion 10, and the area to the right of dotted line 50 is the first strap 21.

In the embodiments of the present application, by setting the lengths of the first strap 21 and the second strap 22 of the abdominal support portion to be unequal, it facilitates the user to perform opening and closing operations on the side of the body or near the side of the abdomen, making the operation more convenient. This is especially beneficial for pregnant women with larger bellies, as side opening and closing are more convenient. Additionally, when the length of the first strap is greater, it provides a larger overall wrapping effect for the abdominal area. At the same time, detaching at the side rather than at the front of the abdomen reduces pressure on the abdomen, thereby improving comfort.

As shown in FIG. 5, in one embodiment, the length of the first strap 21 is greater than the length of the second strap 22. The first strap 21 extends along the right side of the user to the abdominal area when worn, and the second strap 22 extends along the left side of the user to the abdominal area when worn. In other embodiments, the length of the first strap may be less than that of the second strap, which is not limited in the embodiments of the present application.

As shown in FIG. 5, in one embodiment, the first strap 21 comprises a side panel 211 and an abdominal panel 212. The side panel 211 extends along a lateral side of the user's abdomen when worn, and the abdominal panel 212 extends along the user's abdomen when worn. The thickness of the abdominal panel 212 is greater than that of the side panel 211. This design ensures that the abdominal panel 212 provides sufficient cushioning while maintaining adequate breathability for the side panel 211. Additionally, by making the abdominal panel 212 thicker, it provides stronger support in the central abdominal area to distribute gravitational force, while the thinner side panel 211 ensures flexibility for lateral movements and avoids a sense of restraint.

As shown in FIG. 5, in one embodiment, the side panel 211 and the abdominal panel 212 are formed by stitching. The stitching line 213 between the side panel and the abdominal panel is inclined, facilitating the gradual downward curvature of the abdominal panel 212 relative to the side panel 211, thereby enhancing the upward lifting force of the abdominal panel 212. By forming the abdominal panel 212 and the side panel 211 as separate pieces rather than a single cut, the abdominal support belt avoids wrinkles during wear when the abdominal panel 212 changes direction and curves downward, improving the skin-friendly fit and wrapping effect of the abdominal support belt. The stitching line may be treated with double-thread locking or edge-binding techniques to ensure seam strength while keeping the joint smooth and reducing friction and irritation on the skin.

In one embodiment, the waist support portion 10 is embedded with a back support member, the hardness of which is greater than that of other portions of the waist support portion 10. The back support member is made of TPU, rigid silicone, or carbon fiber composite material and is embedded within the interlayer of the waist support portion 10, distributed along the lumbar region to enhance rigid support for the waist while maintaining flexibility in the surrounding areas to accommodate body movements.

In one embodiment, the back support member comprises a plurality of support strips 111 arranged at intervals. The spaced design provides directional support while retaining lateral elasticity, ensuring that the waist remains fitted during bending.

In one embodiment, the support strips 111 extend along a height direction of the user when the abdominal support belt is worn, and the thickness of the support strips 111 gradually decreases from a central portion to both ends. Specifically, the central portion of the support strips 111 is thicker to provide concentrated rigid support for the lumbar region, maintaining the natural curvature of the waist. The gradually thinning design toward the upper and lower ends enhances the flexibility of the edges of the support strips, avoiding pressure from rigid materials on the iliac bones, ribs, or soft tissues during wear. At the same time, the tapered structure reduces the thickness of the sheet material at the upper and lower edges of the abdominal support belt, minimizing the contact area and friction between the edges and the skin. Combined with the smooth transition of the arcuate upper edge, this further eliminates potential skin marks or discomfort caused by pressure. Additionally, the stiffness gradient formed by the gradual thickness change guides the support force to naturally attenuate from the central portion of the waist toward both ends, ensuring core support strength while dynamically adapting the support force to the body's curves. This prevents displacement or fitting failure due to excessive local rigidity, making the abdominal support belt more skin-friendly at the edges.

In one embodiment, the upper edges of the support strips 111 are arcuate. The curvature radius of the arcuate edges matches the natural curve of the human waist, allowing the upper edges of the support strips to smoothly transition to the soft tissues of the waist, preventing straight edges from cutting into the skin or causing discomfort.

In one embodiment, the lower edge of the waist support portion 10 is linear at least at a position corresponding to the user's back. The linear lower edge extends horizontally along the back, fitting the flat area of the human back and avoiding edge lifting or curling caused by an arcuate design, thereby enhancing the stability of the waist support portion 10.

In one embodiment, the first strap 21 extends across the user's abdominal area when worn and is detachably connected to the second strap 22 at a lateral side of the user. This design positions the connection and detachment operations away from the highest point of the abdominal bulge, making it easier for the user to operate while reducing direct pressure on the abdomen during connection or detachment.

In one embodiment, the width of the central portion of the abdominal panel 212 is greater than the widths of both ends. The wider central portion covers the main load-bearing area of the abdomen to increase the support area, while the narrower ends reduce redundant material accumulation on the sides, achieving a balance between concentrated support and flexibility of movement. Optionally, the width of the central portion of the abdominal panel 212 is approximately equal to the width of the user's palm, allowing the support effect of the abdominal panel 212 to mimic the sensation of a hand supporting the abdomen.

In one embodiment, the second strap 22 is provided with a lifting handle 221. The user can apply a lifting force by holding the lifting handle 221 to assist in adjusting the position or tightness of the second strap 22, facilitating the wearing of the abdominal support belt and avoiding direct pressure on the abdomen when pulling the strap.

In one embodiment, the lower edge of the second strap 22 extends obliquely downward from the waist area to the junction 23 where the first strap 21 and the second strap 22 are connected when worn. This design avoids the iliac crest of the human body, improving wearing comfort, while also enhancing the upward lifting force of the abdominal support portion.

In one embodiment, the lower edge of the first strap 21 extends obliquely upward from the abdominal area to the junction 23 where the first strap 21 and the second strap 22 are connected when worn. This design avoids the iliac crest of the human body, improving wearing comfort, while also enhancing the upward lifting force of the abdominal support portion.

In one embodiment, the first strap 21 and the second strap 22 are connected by hook-and-loop fasteners, with one of the first strap 21 and the second strap 22 being provided with a loop surface of the hook-and-loop fastener 222, and the other being provided with a hook surface of the hook-and-loop fastener 222.

In one embodiment, the hook-and-loop fastener 222 on the second strap 22 has a certain length, allowing the detachable connection position between the first strap 21 and the second strap 22 to be adjustable. The user can slide and adjust the connection point between the first strap 21 and the second strap 22 according to changes in waist and abdominal circumference or tightness requirements.

In one embodiment, the hook-and-loop fastener 222 is stitched onto the first strap 21 or the second strap 22 by sewing threads, the sewing threads comprising peripheral sewing lines located at the edge regions of the hook-and-loop fastener and intermediate sewing lines located at the central region of the hook-and-loop fastener. The peripheral sewing lines are stitched along the four sides of the hook-and-loop fastener 222, and the intermediate sewing lines are arranged in an “8” shape, “X” shape, or parallel lines through the central portion of the hook-and-loop fastener 222. This dual fixation prevents the hook-and-loop fastener from peeling or detaching after repeated use, extending its service life.

As shown in FIG. 6, FIG. 6 is a schematic diagram of the layered structure of a sheet material according to an embodiment of the present application. In one embodiment, the abdominal support belt is at least partially made of a sheet material 30, the sheet material 30 comprising an outer layer 31, an inner layer 32, and an adhesive layer 33 interposed between the outer layer 31 and the inner layer 32. The outer layer 31 and the inner layer 32 are bonded together by the adhesive layer 33.

In the abdominal support belt according to the embodiments of the present application, the sheet material 30 used to manufacture the abdominal support belt comprises an outer layer 31, an inner layer 32, and an adhesive layer 33 interposed between the outer layer 31 and the inner layer 32, allowing the sheet material to be freely cut into irregular shapes. Specifically, the adhesive layer tightly bonds the material layers, ensuring no risk of delamination during cutting, thereby enabling the abdominal support belt to be tailored into curved, wavy, or irregular edge contours based on ergonomic requirements. This design allows the abdominal support belt to more precisely conform to the physiological curves of the pregnant user's waist and abdomen. For example, the lower edge of the waist support portion 10 can be cut into a linear shape to fit the flat area of the back, while the edges of the abdominal support portion can be cut with inclined or curved shapes to adapt to the bulging shape of the abdomen. Additionally, this layered structure of the sheet material 30 ensures that the abdominal support belt remains lightweight and discreet while providing strong elasticity and support.

As shown in FIG. 7, FIG. 7 is a schematic diagram of the layered structure of a sheet material according to another embodiment of the present application. In one embodiment, the adhesive layer 33 comprises a first adhesive sublayer 331, a cushioning layer 332, and a second adhesive sublayer 333 sequentially stacked from the inner layer toward the outer layer. By adopting a layered bonding approach, the structural strength of the sheet material is ensured while the cushioning layer provides a flexible buffer space, reducing the rigid pressure on the waist and abdominal areas during wear.

In one embodiment, the first adhesive sublayer 331 and the second adhesive sublayer 333 are adhesive layers. The adhesive layers may use hot-melt adhesive or environmentally friendly binders, bonded to adjacent layers through hot pressing or coating processes, ensuring tight integration between the layers of the sheet material without gaps, thereby avoiding structural looseness or deformation caused by interlayer delamination.

In one embodiment, the cushioning layer 332 is a sponge layer. The sponge layer is made of high-resilience, breathable polyurethane foam or memory foam. Its porous structure further absorbs localized pressure fluctuations during movement while enhancing the breathability of the sheet material, reducing heat buildup.

In one embodiment, the inner layer 32 is provided with ventilation holes. The ventilation holes are distributed in an array on the inner layer, with the hole size and distribution density designed according to ergonomic requirements. By increasing air circulation paths, sweat evaporation is accelerated, preventing skin discomfort caused by heat and moisture accumulation during prolonged wear.

As shown in FIG. 4 and FIG. 5, the present application further provides an abdominal support belt. The abdominal support belt comprises: a waist support portion 10 configured to extend across a waist area of a user and along both lateral sides of the user when worn; and an abdominal support portion connected to the waist support portion 10, configured to be positioned over an abdominal area of the user when worn. The abdominal support portion includes a first strap 21 and a second strap 22 respectively extending along both lateral sides of the waist support portion 10. The first strap 21 and the second strap 22 are detachably connected, and the first strap 21 extends across the user's abdominal area when worn and is detachably connected to the second strap 22 at a lateral side of the user.

In one embodiment, the abdominal support belt comprises a waist support portion 10 and an abdominal support portion. The waist support portion 10 is configured to extend across a waist area of a user and along both lateral sides of the user when worn. Its width and length are designed according to the dimensions of the human waist to fully cover the lumbar region and provide uniform circumferential support. Both sides of the waist support portion 10 are connected to the first strap 21 and the second strap 22 of the abdominal support portion, forming an integrated support structure.

The abdominal support portion is configured to be positioned over an abdominal area of the user when worn and includes a first strap 21 and a second strap 22 respectively extending along both lateral sides of the waist support portion 10. The first strap 21 extends from one side of the waist support portion 10 and passes through the user's abdominal area, while the second strap 22 extends from the other side of the waist support portion 10 to the user's lateral side. The two straps are fixed at the lateral side through a detachable connection structure (e.g., hook-and-loop fasteners, hooks, or snap buttons). This lateral connection design allows the user to operate without bending or relying on external assistance, making it particularly suitable for pregnant women in the late stages of pregnancy who may have limited mobility.

When worn, the first strap 21 extends from the central abdominal area to the lateral side, covering the main bulging area of the abdomen to provide support, while the second strap 22 extends along the lateral side to the connection point with the first strap 21. By adjusting the connection position between the first strap 21 and the second strap 22, the user can customize the tightness according to their waist and abdominal size, ensuring a snug fit and even pressure distribution. Additionally, the detachable connection structure of the first strap 21 and the second strap 22 facilitates quick wearing and removal, while supporting dynamic adjustments to adapt to changes in waist and abdominal shape during pregnancy.

The above design achieves comprehensive support and dynamic adaptation to the waist and abdominal regions through the segmented connection of the waist support portion 10 and the abdominal support portion, as well as the lateral fixation mechanism. This addresses the issues of inconvenient wearing, difficult adjustment, and insufficient fit of traditional abdominal support belts.

The embodiments of the present application provide an abdominal support belt comprising: a waist support portion configured to extend across a waist area of a user and along both lateral sides of the user when worn; and an abdominal support portion connected to the waist support portion, configured to be positioned over an abdominal area of the user when worn. The abdominal support portion includes a first strap and a second strap respectively extending along both lateral sides of the waist support portion. The first strap and the second strap are detachably connected, and the lengths of the first strap and the second strap are unequal. This design allows the user to conveniently open and close the belt at the side or near the side of the body, facilitating user operation.

To make the objectives, technical solutions, and advantages of the present application clearer, the following further describes the present application in detail with reference to the accompanying drawings and embodiments. It should be understood that the specific embodiments described herein are merely intended to explain the present application and are not intended to limit the present application.

The above descriptions are only specific implementations of the present application. It should be noted that, for those of ordinary skill in the art, various modifications and refinements can be made without departing from the principles of the present application. These modifications and refinements should also be regarded as falling within the protection scope of the present application.

What is claimed is:

1. An abdominal support belt for a pregnant user, comprising:
a waist support portion configured to extend across a waist area of the pregnant user and along both lateral sides of the pregnant user when worn;
a back support member disposed on the waist support portion, the back support member including at least one support strip extending along a height direction of the abdominal support belt when worn; and
an abdominal support portion connected to the waist support portion, configured to be positioned over an abdominal area of the pregnant user when worn; wherein the abdominal support portion comprises a first strap and a second strap respectively extending along both lateral sides of the waist support portion, the first strap and the second strap being detachably connected, the length of the first strap is greater than the length of the second strap, the first strap comprises a side panel and an abdominal panel, the side panel extending along a lateral side of the pregnant user's abdomen when worn, the abdominal panel extending along the pregnant user's abdomen when worn, and in an unfolded state of the abdominal support belt, the abdominal panel extends with a downward curvature relative to the side panel, such that when worn, the abdominal panel is configured to provide an upward lifting force to a protruding abdomen of the pregnant user.

2. The abdominal support belt of claim 1, wherein the at least one support strip being progressively curved toward the pregnant user's back from both ends to a central portion thereof when the abdominal support belt is worn; the at least one support strip being a preformed, rigid or semi-rigid member having a body with a predefined continuous curved surface, wherein the member is positioned such that, when the belt is worn and tightened, the predefined continuous curved surface is configured to abut against the pregnant user's back across an area, thereby establishing a load-bearing interface; wherein the at least one support strip comprises at least a first support strip and a second support strip, the first support strip and the second support strip being positioned on opposite lateral sides of the pregnant user's spine when worn, the first support strip being progressively curved from both ends toward the central portion in a direction approaching the spine when the abdominal support belt is worn, and the second support strip being progressively curved from both ends toward the central portion in a direction approaching the spine when the abdominal support belt is worn.

3. The abdominal support belt of claim 1, wherein the at least one support strip being progressively curved toward the pregnant user's back from both ends to a central portion thereof when the abdominal support belt is worn; the at least one support strip being a preformed, rigid or semi-rigid member having a body with a predefined continuous curved surface, wherein the member is positioned such that, when the belt is worn and tightened, the predefined continuous curved surface is configured to abut against the pregnant user's back across an area, thereby establishing a load-bearing interface; wherein the at least one support strip has a thickness that gradually decreases from the central portion to both ends thereof.

4. The abdominal support belt of claim 1, wherein the back support member further comprises a connecting strip bridging adjacent support strips of the at least one support strip.

5. The abdominal support belt of claim 4, wherein the connecting strip comprises at least a first connecting strip and a second connecting strip, the first connecting strip and the second connecting strip being arranged at different positions along the height direction of the abdominal support belt when worn.

6. The abdominal support belt of claim 1, wherein the back support member is embedded within the waist support portion.

7. The abdominal support belt of claim 1, wherein the waist support portion comprises at least a sheet material having an outer layer, an inner layer, and an adhesive layer interposed between the outer and inner layers.

8. The abdominal support belt of claim 7, wherein the adhesive layer comprises a first adhesive sublayer, a cushioning layer, and a second adhesive sublayer sequentially stacked from the inner layer toward the outer layer.

9. The abdominal support belt of claim 8, wherein the back support member is embedded within the cushioning layer.

10. The abdominal support belt of claim 8, wherein the cushioning layer comprises a sponge layer.

11. The abdominal support belt of claim 7, wherein the inner layer is provided with ventilation holes.

12. The abdominal support belt of claim 1, wherein the abdominal support portion is configured to overlay a lower abdominal area of the pregnant user when worn.

13. The abdominal support belt of claim 1, wherein the back support member is disposed on an exterior surface of the waist support portion.

14. The abdominal support belt of claim 1, wherein the at least one support strip has an arcuate upper edge, the at least one support strip has an arcuate lower edge.

15. (canceled)

16. (canceled)

17. (canceled)

18. (canceled)

19. (canceled)

20. (canceled)

21. (canceled)

22. (canceled)

23. (canceled)

24. (canceled)

25. The abdominal support belt of claim 1, the thickness of the abdominal panel being greater than the thickness of the side panel.

26. The abdominal support belt of claim 1, a width of a central portion of the abdominal panel is greater than widths of both ends of the abdominal panel.

27. The abdominal support belt of claim 1, wherein the second strap is provided with a lifting handle configured to allow the user to apply a lifting force to adjust the position or tightness of the second strap.
